SEQUENCE LISTING (17 sequences)

SEQ ID NO: 1 - protein, 6 aa, Artificial sequence
  CAPRI activating peptide (pseudo-RACK1)
CVEAWD

SEQ ID NO: 2 - DNA, 18 bp, Artificial sequence
  Coding sequence for SEQ ID NO 1 CVEAWD (667-684 of Genbank AY029206)
tgcgtggaggcctgggac

SEQ ID NO: 3 - protein, 13 aa, Artificial sequence
  CAPRI inhibitory peptide (C2-2)
KDRNGTSDPFVRV

SEQ ID NO: 4 - DNA, 39 bp, Artificial sequence
  Coding sequence for SEQ ID NO 3 KDRNGTSDPFVRV (520-558 of Genbank AY029206)
aaggaccgcaatggcacatctgaccccttcgtccgagtg

SEQ ID NO: 5 - protein, 9 aa, Artificial sequence
  CAPRI inhibitory peptide (C2-4)
SCYPRWNET

SEQ ID NO: 6 - DNA, 27 bp, Artificial sequence
  Coding sequence for SEQ ID NO 5 SCYPRWNET (601-627 of Genbank AY029206)
tcatgctacccacgctggaatgagacg

SEQ ID NO: 7 - protein, 6 aa, Artificial sequence
  RASAL activating peptide (pseudo-RACK1)
RVELWD

SEQ ID NO: 8 - RNA, 18 nt, Artificial sequence
  Coding sequence for SEQ ID NO 7 RVELWD (882-899 of Genbank NM_004658)
cggguggagcucugggac

SEQ ID NO: 9 - protein, 9 aa, Artificial sequence
  RASAL inhibitory peptide (C2-4)
TRFPHWDEV

SEQ ID NO: 10 - RNA, 27 nt, Artificial sequence
  Coding sequence for SEQ ID NO 9 TRFPHWDEV (816-842 of GenBank NM_004658)
acucgcuucccgcacugggaugaagug

SEQ ID NO: 11 - protein, 13 aa, Artificial sequence
  RASAL inhibitory peptide (C2-4)
RDISGTSDPFARV

SEQ ID NO: 12 - RNA, 45 nt, Artificial sequence
  Coding sequence for SEQ ID No 11 RDISGTSDPFARV (729-773 of GenBank NM_004658)
gcucccagagacaucucuggcacaucugacccauuugcacgugug

SEQ ID NO: 13 - protein, 91 aa, Artificial sequence
  PKCbeta  C2B domain peptide
LIVLVRDAKNLVPMDPNGLSDPYVKLKLIPDPKSESKQKTKTIKCSLNPEWNETFRFQLKESDKDRRLSVEIWDWDLTSRNDFMGSLSFGI

SEQ ID NO: 14 - protein, 85 aa, Artificial sequence
  CAPRI peptide
LRCSVLEARDLAPKDRNGTSDPFVRVRYKGRTRETSIVKKSCYPRWNETFEFELQEGAMEALCVEAWDWDLVSRNDFLGKVVIDV

SEQ ID NO: 15 - protein, 11 aa, Artificial sequence
  GAP1{circumflex over ( )}m segment peptide
TVCQQLVVHIK

SEQ ID NO: 16 - protein, 44 aa, Artificial sequence
  GAP1{circumflex over ( )}m segment peptide
LPLINGQSCDPYATVSLVGPSRNDQKKTKVKKKTSNPQFNEIFY

SEQ ID NO: 17 - protein, 36 aa, Artificial sequence
  GAP1{circumflex over ( )}m segment peptide
FQVEEEDIEKLEIRIDLWNNGNLVQDVFLGEIKVPV

(19) United States
(12) Patent Application Publication
(10) Pub. No.: US 2007/0128665 A1
(43) Pub. Date: Jun. 7, 2007

(54) Methods for identifying compounds interacting with small membrane-bound gtp-ases

(76) Inventor: Peter Lockyer, Babraham Cambridge (GB)

Correspondence Address: GOODWIN PROCTER LLP;PATENT ADMINISTRATOR, EXCHANGE PLACE, BOSTON, MA, 02109-2881, US

(21) Appl. No.: 10/560,956

(22) PCT Filed: Apr. 26, 2004
(86) PCT No.: PCT/GB04/01765

§ 371 Date: Jul. 24, 2006

(30) Foreign Application Priority Data
Jun. 26, 2003 (GB) 03149804

(51) Int. Cl. G01N 33/567; G01N 33/53

(52) U.S. Cl. 435/7.2

(57) ABSTRACT

A method for identifying a compound capable of promoting deactivation of, or inhibiting activation of, a membrane bound active small GTPase, comprising incubating a live cell expressing the small GTPase and having a small GTPase specific reporter thereof in the presence of a test compound and monitoring association of the reporter with the membrane bound small GTPase, wherein a change in association of the reporter with the membrane bound small GTPase is indicative that the test compound is capable of promoting deactivation of, or inhibiting activation of, the membrane bound small GTPase.

### TECHNICAL FIELD

This invention relates to methods for identifying compounds, in particular to methods for identifying compounds that deactivate small GTPase proteins or inhibit activation of small GTPase proteins.

### BACKGROUND

Small GTPases, also called small GTP-binding proteins, are binary molecular switches, cycling between an inactive GDP-bound form and an active GTP-bound form usually at a cell membrane because they are post-translationally modified by a lipid moiety. In the active form, small GTPases bind to, and stimulate, specific effector pathways that are implicated in a plethora of cellular pathways that regulate a very diverse set of cellular processes, from cell growth and maintenance to cell death. The relative fraction of small GTPase in an active, GTP-bound conformation depends on the rates of GDP dissociation and GTP hydrolysis. Guanine nucleotide exchange factors (GEFs) bind to small GTPases and accelerate the rate of GDP dissociation and thus promote activation of the small GTPase. Deactivation is stimulated by the binding of GTPase-activating proteins (GAPs) that promote the intrinsic GTPase activity so that the small GTPase converts to the inactive GDP-bound form.

The small GTPases are divided into groups according to homology, and to a lesser extent function. The Ras superfamily of small GTPases includes the Ras, Rho, Ran, Arf/Sar1 and Rab/YPT1 subfamilies.

Mutant, constitutively active and hyperactive normal small GTPases are associated with development of tumours. Current tumour treatments depend on surgery and chemo/radiotherapy. Small GTPases are thus attractive targets for development of anti-tumour therapies.

Ras small GTP-binding proteins are of particular interest in research for tumour therapies. Ras proteins regulate cell growth and differentiation in a pathway from transmembrane receptors to mitogen activated protein kinases (MAPKs) and the control of gene expression. Ras is a particularly important small GTPase in relation to human disease because activating mutations in the human ras oncogenes contribute to the formation of approximately 30% of human malignancies, in which tumours contain constitutively active, GTP-bound mutant forms of oncogenic Ras [3]. These mutant forms of Ras are capable of binding Ras GAPs but they are resistant to their action, so that the intrinsic GTPase activity is not enhanced and Ras remains trapped in the GTP-bound active conformation. Other small GTPases, including some that are downstream of Ras signalling, may also contribute to tumorigenesis and metastasis. For example, Rho family small GTPases are frequently stimulated by active Ras and are involved in regulating the cell cytoskeleton and controlling cell motility.

Three human Ras genes encode four forms of Ras: H-Ras, N-Ras, K-Ras4A and K-Ras4B. The initial Ras gene translation products are cytosolic proteins but they ultimately become membrane localised because post-translational modification of the CAAX sequence found at the C terminus of all Ras proteins results in acquisition of a membrane targeting signal. Post-translational modifications include farnesylation of the CAAX cysteine, followed by proteolytic removal of the AAX amino acids and methylesterification of the α-carboxyl group of the C terminal prenyl cysteine. N-Ras and H-Ras are further modified by palmitate at one or two cysteines respectively adjacent to the terminal cysteine. In K-Ras a polylysine motif provides the plasma membrane targeting signal.

Ras operates as a binary molecular switch, cycling between inactive GDP-bound form and an active GTP-bound form at the membrane [1]. It transduces signals from cell surface receptors into the cytoplasm via effector pathways that regulate cell growth, differentiation and apoptosis [2]. In vitro, it exhibits slow rates of GDP dissociation and GTP hydrolysis, thus the relative fraction of cellular Ras in an active conformation depends on the rates of these two reactions. Guanine nucleotide exchange factors (GEFs) bind to Ras and markedly accelerate the rate of GDP dissociation. In contrast, deactivation requires the binding of GTPase-activating proteins (GAPs) that significantly enhance the intrinsic Ras GTPase activity. Overall the spatio-temporal regulation of GEFs and GAPs coordinates Ras signalling events but this integration of Ras activation/deactivation is highly complex as there are multiple Ras GEFs and Ras GAPs that are stimulated or inhibited, depending on the nature of the signal. The activation state of Ras is critical in determining the ability of Ras to cause transformation.

Ras genes are proto-oncogenes and oncogenic forms of mutant Ras are locked in the GTP-bound, active state, immune to the action of GAPs [1, 2]. This renders them constitutively active and able to transform some mammalian cells. Other genetic lesions, for example in the Ras GAP neurofibromin (responsible for the genetic disease neurofibromatosis type 1) [4] or oncogene products upstream of Ras [5-7], can lead to hyperactive Ras signalling. Alternatively, expression of abnormally high levels of normal Ras may also contribute to transformation due to hyperactive Ras signalling [8]. For these reasons Ras-activated pathways and anti-Ras strategies are being intensively targeted by pharmaceuticals.

The most hopeful anti-Ras pharmaceutical strategy currently available and under continuing development are the famesyltransferase inhibitors (FTIs; Omer, C. A & Kohl, N. E. CA₁A₂X-competitive inhibitors of farnesyl-transferase as anti-cancer agents. TIPS (1997) V18:437-445).

Tumour cells are predisposed to becoming drug resistant due to a high mutational frequency. For example, FTI resistance has been observed, both in cell culture and in animals. These strategies target the oncogene to block activity e.g. an active site or post-translational modification. Thus, it can be relatively simple for mutations to retain or alter activity but reduce inhibitor efficacy. In addition, treatments such as FTIs are not specifically targeted to Ras only. They have unpredictable affects on other enzymes and proteins and are not highly selective.

Ras GAPs (GTPase-activating proteins) switch-off activated Ras and it has been shown that loss of Ras GAP function can lead to cancer. Such is the case for the NF1 tumour suppressor gene responsible for neurofibromatosis type 1 [4]. In particular, single point mutations affecting NF1 function have been detected in NF1 patients, indicating that inactivation of Ras GAP activity results in manifestation of the disease (Upadhyaya et al. Neurofibromatosis Type 1 from Genotype to Phenotype (Oxford: BIOS Scientific Publishers Limited 1998).

Several assays to detect activation of small GTPases have been developed.

Mochizuki et al. (Spatio temporal images of growth factor induced activation of Ras and Rap1 Nature 411, 1065-1068 (2001)) describe a fluorescent resonance energy transfer (FRET) assay for growth factor induced activation of Ras and Rap1. The Ras reporter, Raichu-Ras, (Raichu standing for Ras and interacting protein chimeric unit) has been engineered as a single chimeric protein that consists of a terminal yellow fluorescent protein (YFP) and a terminal cyan fluorescent protein (CFP) flanking a peptide consisting of H-Ras and the Ras-binding domain (RBD) of Raf. FRET involves the transfer of energy from a donor fluorescent molecule to an acceptor, which then emits its own fluorescence. Importantly this process only occurs when the two fluorescent proteins are very close to one another. In serum-starved, unstimulated cells Ras is inactive, the fluorescent proteins are widely spaced apart, and the emission profile of CFP peaks at 475 nm upon excitation at 433 nm (close to the excitation maxima of CFP). On cellular stimulation Ras is activated by becoming GTP-bound, which induces Raichu-Ras to change conformation as a result of the RBD interacting with GTP-Ras. The two fluorophores are now in close proximity, so the energy that is emitted by CFP is partially captured by YFP, which emits light at 527 nm. Using computer-enhanced time-lapse video microscopy the ratio of emission at 527 nm and 475 nm can be calculated in order for the spatio-temporal dynamics of Ras activation to be measured. Similar intramolecular FRET probes have been developed for Rap and Rho family members.

Chiu, V. K. et al. (Ras signalling on the endoplasmic reticulum and Golgi Nature Cell Biology 4, 343-350 (2002)) disclose a fluorescent probe that reports active Ras in living cells. The probe is based on the Ras binding domain (RBD) of the Ras effector Raf-1, tagged at the amino terminus with green fluorescent reporter. Activation of Ras following stimulation is detected as recruitment of the fluorescent reporter to certain intracellular membranes.

WO02/052272A2 describes a method for investigating compounds that affect the activity of the oncogenic Ras mutants (e.g. G12V Ras) in which the membrane localisation signal (CAAX box) is absent and a nuclear localisation signal (NLS) is substituted. This Ras protein is not membrane bound, instead it exists in soluble form in the nucleoplasm. A second protein consists of a fluorescent probe for the first oncogenic Ras protein (e.g.GFP-RBD) with a nuclear export sequence (NES). When the first and second proteins are expressed together the soluble nuclear oncogenic Ras pulls the GFP-RBD-NES probe into the nucleus as a marker of the interaction. The NES sequence on the probe avoids a build up of the fluorescent probe in the nucleoplasm, ensuring that the probe is exported from the nucleus when it is not bound to active Ras.

A Ras-GTP pull down assay has been used [10] in which cells are lysed and the cell extract passed down a column on which the Ras binding domain (RBD) of Raf-1 has been immobilised. After washing, active Ras-GTP is eluted from the column and can be quantified to determine the level of active Ras (Ras-GTP) in cells. This assay allows determination of the level of active Ras, but gives no indication of the. spatio temporal activation of Ras.

In vitro cell free assays for activation of certain GAPs are known. However, these assays are not applicable to all GAPs. Certain GAPs such as CAPRI cannot be assessed as they are not activated in these cell free in vitro methods.

The present inventors identified a human gene at 7q22 which is a strong candidate for a tumour suppressor gene based on its Ras GAP function [10]. The gene encodes a calcium promoted Ras inactivator (CAPRI), calcium is a universal second messenger critical for cell growth and intimately associated with many Ras-dependent cellular processes, such as proliferation and differentiation [1,2,9]. The importance of the calcium ion Ca²⁺, as a second messenger that regulates the ability of Ca²⁺ effector proteins to modulate Ras signalling is an emerging theme in cell biology [9].

CAPRI is a member of the human GAP1 family of Ras GAPs (GAP1^{IP4BP}, GAP1^{m}, RASAL) that have a similar domain structure comprising of tandem C2 domains (C2A and C2B), a central GAP-related domain (GRD) contiguous with a pleckstrin homology domain (PH domain) and Tec kinase homology domain (TH) near the C-terminus (FIG. 1). Within the human GAP1 family, RASAL (Allen, Chu et al. 1998) is most closely related to CAPRI with 59% identity at the primary amino acid sequence level.

An important advance towards greater understanding of the complex coordination within the Ras signalling network is the spatio-temporal analysis of signalling events in vivo. In resting cells CAPRI is cytosolic and inactive. Following a stimulus that elevates intracellular calcium, CAPRI is translocated to the membrane, and is believed to undergo a conformation change and activation. Ras GAPs such as p120 Ras GAP and GAP1^{m} are basally active in the cytosol despite having intrinsic mechanisms for translocation to the plasma membrane.

Activated CAPRI inhibits the Ras/mitogen-activated protein kinase (MAPK) pathway by enhancing the intrinsic GTPase activity of Ras, resulting in deactivation [10]. Analysis of the spatio-temporal dynamics of CAPRI indicates that calcium regulates the GAP by a fast C2 domain-dependent translocation mechanism [10]. Analysis was carried out in a whole cell assay in which CAPRI and CAPRI deletion mutants tagged with a green fluorescent protein (GFP) were expressed. Agonist-dependent increases in intracellular Ca²⁺ induced a rapid translocation to the plasma membrane and activation of CAPRI [9 and data unpublished]. This recruitment of CAPRI to the plasma membrane was detected because the GFP linked to CAPRI resulted in acquisition of a fluorescent signal at the plasma membrane.

A role for intracellular calcium in the activation of Ras has been previously demonstrated, e.g. via the non-receptor tyrosine kinase PYK2 and by calcium/calmodulin-dependent guanine nucleotide exchange factors (GEFs) such as Ras-GRF [9], however until the discovery of CAPRI there was no known calcium-dependent mechanism for direct inactivation of Ras.

Prior art methods have concentrated on detecting activation of small GTPases and dissecting the signalling pathways that result in activation. It is an aim of the present invention to provide a methods for identification of compounds, particularly those that act intracellularly, that inhibit activation, or promote deactivation of membrane bound small GTPases, such as Ras.

### DETAILS OF THE INVENTION

The present invention provides a method for identifying a compound capable of promoting deactivation of, i.e. switching off, a membrane bound active small GTPase, comprising:
incubating in the presence of a test compound a live cell expressing the membrane bound small GTPase and having a small GTPase specific reporter thereof, and,
monitoring association of the reporter with the membrane bound small GTPase,

wherein a change in association of the reporter with the membrane bound small GTPase is indicative that the test compound is capable of promoting deactivation of, i.e. switching off, the membrane bound active small GTPase.

This method can be used to detect compounds capable of promoting deactivation of, i.e. switching off, a membrane bound active small GTPase, e.g. by acting directly on the small GTPase to enhance its intrinsic GAP activity, or indirectly, by stimulating a GAP.

The invention provides a method for identifying a compound capable of enhancing the intrinsic GTPase activity of an membrane bound active small GTPase, comprising:
incubating in the presence of a test compound a live cell expressing the membrane bound small GTPase and having a small GTPase specific reporter thereof, and,
monitoring association of the reporter with the membrane bound small GTPase

wherein a change in association of the reporter with the membrane bound small GTPase is indicative that the test compound is capable of enhancing the intrinsic GTPase activity of the membrane bound small GTPase.

The method for detecting compounds that enhance the intrinsic GTPase activity of a small GTPase, and thereby convert it from the active form to the inactive form, is especially useful for detecting compounds that inhibit normal and hyperactive normal small GTPases, and those that inhibit constitutively active mutants, e.g. oncogenic forms, such as oncogenic Ras, that are otherwise locked in an active GTP-bound state.

In methods of the invention, a change in association of the reporter with the membrane bound small GTPase may be dissociation from the membrane of a reporter specific for the active form of the small GTPase, or association with the membrane of a reporter specific for the inactive form of the small GTPase. Preferably the change in association of the reporter with the membrane bound small GTPase is dissociation of a reporter specific for the active form of the small GTPase.

The present invention provides a method for identifying a compound capable of inhibiting activation of a membrane bound small GTPase, i.e. preventing a membrane bound small GTPase from being switched on, comprising:
incubating in the presence of a test compound a live cell expressing the membrane bound small GTPase and having a small GTPase specific reporter thereof and optionally overexpressing a GEF that activates the membrane bound small GTPase, and,
monitoring association of the reporter with the membrane bound small GTPase,

wherein a change in the association of the reporter with the membrane bound small GTPase is indicative that the test compound is capable of inhibiting activation of the membrane bound small GTPase, i.e. preventing the membrane bound small GTPase from being switched on.

This method is useful for identification of compounds that block the upstream pathway. It relies on the intrinsic GTPase activity to run-down the active (GTP bound) membrane bound small GTPase. It is particularly appropriate for identification of compounds that inhibit activation of normal and hyperactive normal small GTPases.

The present invention provides a method for identifying a compound capable of inhibiting GTP loading on a membrane bound small GTPase, comprising:
incubating in the presence of a test compound a live cell expressing the membrane bound small GTPase and having a small GTPase specific reporter thereof and optionally overexpressing a GEF that activates the small GTPase, and,
monitoring association of the reporter with the membrane bound small GTPase,

wherein a change in the association of the reporter with the membrane bound small GTPase is indicative that the test compound is capable of inhibiting GTP loading.

The present invention provides a method for identifying a compound capable of inhibiting GTP loading on a membrane bound small GTPase by directly blocking guanine nucleotide exchange factor-stimulated GDPIGTP exchange, or by inhibiting upstream pathways that lead to the activation of the exchange factor, comprising:
incubating in the presence of a test compound a live cell expressing the membrane bound small GTPase and having a small GTPase specific reporter thereof, and,
monitoring association of the reporter with the membrane bound small GTPase,

wherein a change in the association of the reporter with the membrane bound small GTPase is indicative that the test compound is capable of inhibiting GTP loading.

Methods of the invention for identifying a compound capable of inhibiting activation of a membrane bound small GTPase can be used to identify a compound which has an inhibitory effect on the membrane bound small GTPase because it is capable of inhibiting GTP loading on the membrane bound small GTPase. GTP loading may be prevented by directly blocking the association of the GEF with the membrane bound small GTPase (thereby blocking guanine nucleotide exchange factor-stimulated GDP/GTP exchange) or promoting an interaction between a membrane bound small GTPase and a GDP dissociation inhibitor (GDI). Alternatively, GTP loading may be indirectly inhibited by blocking an upstream signal that activates GEF function. In such methods, a compound capable of modulating activity of a specific GEF can be evaluated, optionally, using a cell which has been transformed with a specific GEF resulting in overexpression of the GEF, alternatively, natural or engineered mutants in which a specific GEF is overexpressed may be employed.

The invention provides a method for identifying a compound capable of modulating interaction of a membrane bound small GTPase with a binding partner, comprising:
incubating in the presence of a test compound a live cell expressing the membrane bound small GTPase and having a small GTPase specific reporter thereof, and
monitoring association of the reporter with the membrane bound small GTPase

wherein a change in association of the reporter with the membrane bound small GTPase is indicative that the test compound is capable of modulating the interaction between the membrane bound small GTPase and its binding partner.

The test compound may promote interaction of the membrane bound small GTPase with the binding partner or may inhibit interaction of the membrane bound small GTPase with the binding partner.

The binding partner may be, for example, an effector of the small GTPase or a peptide derived from the effector, optionally linked to a detectable marker. The binding partner may be the reporter specific for the membrane bound small GTPase.

This method permits detection of inhibitors of the membrane bound small GTPase-reporter interaction. When the reporter used is derived from an effector of the small GTPase, the method can be used to identify compounds that disrupt the interaction between the small GTPase and its effector, which compounds can act as blockers of downstream signalling. For example, this method would permit detection of inhibitors of the Ras-GTP/GFP-RBD reporter interaction, which can act as blockers of downstream Ras-Raf signalling. As B-Raf is an oncogene, the Ras-Raf-MAPK pathway is generally considered to promote DNA synthesis and cell proliferation, therefore inhibitors of this pathway are potentially therapeutically useful.

The present invention provides a method for identifying a compound capable of promoting deactivation of, i.e. switching off, a membrane bound active Ras, comprising:
incubating in the presence of a test compound a live cell expressing a Ras (which is membrane bound) and having a specific reporter thereof, preferably GFP-RBD or a derivative thereof, and,
monitoring association of the reporter, preferably GFP-RBD or a derivative thereof, with the membrane bound active Ras,

wherein a dissociation of the reporter from the membrane bound active Ras is indicative that the test compound is capable of promoting deactivation of, i.e. switching off, the membrane bound active Ras.

Methods of the invention are beneficial compared to prior art methods.

Chiu et al (supra) describes an assay for activation of Ras and but does not provide a method for identification of compounds that deactivate active Ras.

The (FRET) assay using a single chimeric protein molecule as described by Mochizuki et al reports only intrinsic activity and is not suitable to assay endogenous small GTPase activity in cells such as a tumour cells expressing oncogenic Ras or hyperactive normal Ras. The chimeric molecule reports the balance between GEF and GAP activities on the membrane to which it is targeted. It does not monitor active Ras per se. In this assay the post-translational modification is from K-Ras, therefore, the reporter is H-Ras and is targeted to where K-Ras would normally reside. This places a chimeric Ras molecule in a potentially artificial environment, which is different to the normal localisation of H-Ras, making the physiological basis of activity measurements difficult to interpret. Changes in FRET signals are small, so computer-enhancement, multiple wavelength measurements and detailed analysis are required to ensure that the signal detected is a bona fide FRET signal. Furthermore, the assay may lack sensitivity. This is a complicated assay, and FRET assays cannot easily be adapted for high throughput screening in live cells.

The assay described in WO 02/052272A2 uses an engineered oncogenic form of Ras which is soluble in the nucleoplasm. Nuclear localisation is useful because a high signal to noise ratio is generated from nuclear fluorescence compared to cytosolic fluorescence. However, Ras, whether it be normal, normal hyperactive or mutant oncogenic Ras is normally a membrane bound protein, so the assay scenario using a soluble form is very artificial. It cannot, for example, be used to identify compounds that influence upstream signals that result in modulation of Ras activity. Furthermore, because the engineered Ras is soluble, it may not adopt a conformation comparable to that of the membrane bound form, so any compounds found to interact or influence the activity of soluble Ras may not interact in the same manner, if at all, with membrane bound Ras. A further disadvantage of this assay compared to methods of the invention is that cells must be transformed with a construct for expression of the soluble oncogenic Ras. In contrast, in the methods of the invention, modulation of the endogenous activity of Ras can be assessed. Thus methods of the invention can be applied, for example, to cells taken from primary human tumours in patients, allowing sensitivity of the human tumour cells to various agents to be determined, which may be important in selection of therapeutic strategy.

Methods of the invention provide a simple, sensitive, robust means for identification of compounds that inhibit activation of, or deactivate, membrane bound small GTPases. The methods can readily be adapted for high throughput screening.

The terms small GTPase and small GTP-binding protein are used interchangeably. The small GTPase can be a Ras superfamily GTPase, in particular a Ras, Rho, Ran, Arf/Sar1, or Rab/YPT1 subfamily GTPase. In preferred methods of the invention, the small GTPase is a Ras GTPase.

A particular small GTPase will be membrane bound at a particular membrane or group of membranes where it will be biologically active. Membrane bound small GTPases are found at one or more of the following membrane locations: the plasma membrane, Golgi apparatus membrane, endomembrane, lysosome, mitochondrial membrane, outer nuclear membrane, inner nuclear membrane, endoplasmic reticulum, sarcoplasmic reticulum and/or a membrane of transport and/or secretory vesicles. Ras is found at the plasma membrane, Golgi membranes, endoplasmic reticulum (E.R.) and on vesicular membranes between the E.R., Golgi and plasma membrane.

The active membrane bound small GTPase may be a mutant, constitutively active form, which may be oncogenic. Alternatively, the membrane bound small active GTPase may be a normal active or hyperactive form. Hyperactive membrane bound small GTPases are normal but are hyperactive, e.g. due to inappropriate, overactive, upstream signalling, such as by another oncogene, e.g. a receptor tyrosine kinase, or due to loss of a GAP which would normally deactivate the membrane bound small GTPase, e.g. neurofibromin for Ras.

In methods of the invention the membrane bound small GTPase monitored is preferably active Ras (Ras-GTP), which can be oncogenic Ras, hyperactive normal or active normal Ras.

In a method of the invention where the membrane bound small GTPase is a normal active form, this may be activated, for example by agonist stimulation of the cell, e.g. by stimulation using growth factors, before and/or during incubation with the test compound.

A change in association of the reporter with the membrane bound small GTPase can be dissociation of the reporter from the membrane bound small GTPase and thus from the membrane, or an association or recruitment of the reporter to the membrane bound small GTPase, and thus to the membrane.

Reporters capable of specific binding to either an active (on) small GTPase or to an inactive (off) small GTPase can be used in methods of the invention. In methods where the reporter binds specifically to an active small GTPase (i.e. the reporter is an active small GTPase specific reporter), inhibition of activation, or stimulation of deactivation, of the small GTPase will be detected as dissociation of the reporter from the membrane. In methods where the reporter binds specifically to an inactive small GTPase, (i.e. the reporter is an inactive small GTPase specific reporter) inhibition of, or failure to activate, the small GTPase will be detected as association of the reporter with the membrane.

A reporter which binds specifically to an active form of a membrane bound small GTPase has higher affinity for the active form than the inactive form of the membrane bound small GTPase such that it can be used to distinguish between the two forms. Similarly, a reporter which binds specifically to an inactive form of a membrane bound small GTPase has higher affinity for the inactive form compared to the affinity that it has for the active form of the membrane bound small GTPase, allowing the reporter to distinguish between the two forms.

The reporter preferably comprises a small GTPase specific binding moiety and a detectable marker moiety.

The small GTPase specific binding moiety is preferably a peptide sequence from an effector of the small GTPase, or derivative thereof, which may optionally have one or more mutations (one or more amino acid substitutions, deletions or additions) that increase the affinity of the peptide for the small GTPase relative to the affinity of the wild type effector or wild type effector peptide for the small GTPase.

In a preferred aspect of the invention the small GTPase monitored is an active Ras and the small GTPase-specific binding moiety is an active-Ras-specific-binding moiety. The active-Ras specific binding moiety is preferably Raf-1-RBD or a derivative thereof capable of binding to active Ras. Suitable derivatives include amino acids 51 to 131 of the human Raf-1-RBD (Raf-1-RBD 51-131, also referred to as “RBD”), and amino acids 51 to 200 of the human Raf-1-RBD (Raf-1-RBD 51-200) which includes the cysteine rich domain (CRD) of human Raf 1 (residues 139-184).

In a preferred aspect of the invention the small GTPase monitored is any active Rho family member and the small GTPase specific binding moiety is an active Rho specific binding moiety for many Rho family members. Active Rho family members are important for cell motility and control of the cytoskeleton. The active Rho family specific binding moiety is preferably the Rhotekin binding domain, or a derivative thereof. Rhotekin binds to active Rho family GTPases such as RhoA, RhoB, Rac and Cdc42.

In a preferred aspect of the invention the small GTPase monitored is active Cdc42 and the small GTPase specific binding moiety is an active Cdc42 specific binding moiety. Active Cdc42 is a Rho family member important for cell motility. The active Cdc42 specific binding moiety is preferably WASP-CRIB (Kim, S. H., et al. (2000) J. Biol. Chem, 275, 36999-37005) or a derivative thereof.

Alternatively, the small GTPase monitored is Rac and the small GTPase specific binding moiety is an active Rac specific binding moiety, preferably the CRIB domain from P21 activated kinase (PAK) (Srinivasan, S., (2003) J. Cell Biol., 160, 375-385), or a derivative thereof.

In another preferred aspect of the invention, the small GTPase monitored is active Rap1 and the small GTPase specific binding moiety is an active Rap1 specific binding moiety. Rap 1 is a Ras superfamily member important in cell proliferation, cell motility and cell adhesion. The active Rap1 specific binding moiety is preferably a peptide of RaIGDS or a derivative thereof (Bivona et al (2004) J. Cell Biol.; 164(3):461-70).

The detectable reporter is preferably a protein. The reporter may be transiently introduced into the cell e.g. by transfection or may be integrated and stably expressed within the cell.

Cells used in the methods of the invention have a detectable reporter specific for the membrane bound small GTPase of interest. In certain embodiments of the invention, the detectable reporter is a protein expressed within the cell. The cell can be engineered to express the detectable reporter protein from stably integrated nucleic acid. For ectopic expression, the cell can be stably or transiently transfected with nucleic acid encoding the detectable reporter protein, suitably the nucleic-acid encoding the detectable reporter protein is comprised within an expression vector.

In alternative embodiments of the methods, the cells do not express the detectable reporter and instead the cells have the detectable reporter because it is introduced into the cell for purposes of conducting the assay, e.g. by permeabilisation, by using lipid reagents, or microinjection. Thus as an alternative to expressing the detectable reporter within the cell, the detectable reporter can be introduced into the cell.

The detectable marker moiety may be a luminescent or fluorescent protein, but is preferably a fluorescent protein. In preferred methods, the reporter is labelled with a fluorescent marker.

A suitable reporter for use in methods where the reporter is expressed within the cell, or in methods where the reporter is introduced into the cell, is a protein chimera having a small GTPase specific reporter moiety and a fluorescent protein moiety.

The fluorescent protein can be a red, orange, yellow, yellow-green, green-yellow, green blue or cyan fluorescent protein. Most preferably the fluorescent protein is monomer. Preferably the reporter has only a single detectable marker, preferably a single luminescent or fluorescent protein, most preferably a single monomeric red, orange, yellow, yellow-green, green-yellow, green, blue or cyan fluorescent protein. The fluorescent protein can be a wild type, enhanced, destabilised enhanced or red-shift or folding mutant fluorescent protein.

As an alternative to using a single fluorescent protein marker system, a fluorescence resonance energy transfer (FRET) method using two fluorescent proteins may be used to detect the location of the reporter within the cell, e.g. FRET between a plasma membrane-localised fluorophore and a small GTPase specific reporter having a fluorescent marker, e.g. a targeted CFP having a lipid group or transmembrane protein for targeting to the membrane and having a YFP-reporter, the YFP-reporter can be endogenously or ectopically expressed within the cell or introduced into the cell.

Other suitable reporters are those in which a small GTPase specific protein moiety is either expressed within the cell, or introduced into the cell and is labelled in vivo, i.e. within the cell, with a fluorescent moiety which is introduced into the cell. Such reporters include: a reporter in which the C-terminus of the reporter is fused to ^{W160}hAGT (O⁶-alkylguanine-DNA alkyltransferase) which is fluorescently labelled following a reaction with O⁶-benzylguanine fluorescein (BGFL); and a detectable reporter in which the reporter a tetracysteine motif is added to the N— or C-terminus of the reporter and to which a bi-arsenic fluorophore is covalently linked (‘FIAsH labelling’).

Other fluorescent reporters cannot be expressed within the cell, but are suitable for use in methods of the invention where the small GTPase specific reporter is introduced into the cell for example a reporter in which the small. GTPase specific reporter moiety is labelled with a fluorophore, for example a small organic fluorophore, e.g. fluorescein or rhodamine or a cyanine, such as a Cy Dye™ Fluor (Amersham Biosciences); or a quantum dot (Q dot®, Quantum Dot Corporation).

In a preferred embodiment in which a reporter is introduced into a cell, the small GTPase specific reporter is labelled with a fluorescent marker which is one or more quantum dot. Quantum dots are tiny particles made from nanocrystal semiconductor materials, such as cadmium selenide. Dots of different sizes absorb UV light but then re-emit light at a different wavelength, usually at visible frequencies. The size of the dot determines the colour of light that it emits: a 2 nanometre dot emits green light, while a 5 nanometre dot emits red light. The reporter can be directly labelled with the quantum dot and introduced into the cell, alternatively the reporter can be labelled with the quantum dot by using a biotinylated reporter and streptavidin coated quantum dot both of which are introduced into the cell. As a further alternative using a quantum dot as the detectable marker, the reporter can be expressed within the cell as a chimera with avidin and a biotin-labelled quantum dot can be introduced into the cell.

A detectable reporter according to present invention is generally applicable for detecting the membrane bound small GTPase and reporting its activation state, whether by fluorescence, luminescent or other detection techniques, depending on the detectable marker employed.

In preferred methods of the invention, the detectable reporter is a membrane bound small GTPase specific reporter, suitably an active membrane bound specific GTPase reporter, labelled with a fluorescent protein.

Fluorescent protein chimeras comprising the membrane bound small GTPase specific reporter and a fluorescent protein, e.g. green fluorescent protein, can be used as genetically-encodable reporters of small GTPase activation status and are thus suitable for use in methods of the invention.

Constructs encoding the detectable reporter can be transfected into cell lines by standard techniques e.g. electroporation, Ca²⁺ phosphate, lipofection, gene gun. Recombinant retroviruses, adenoviruses or lentiviruses can also be used to introduce genetic material encoding the reporter into cells by infection. Selection of cells expressing a fluorescent protein (FP) chimeric reporter can be made by FACS, or where a vector is used, an antibiotic resistance gene carried by the vector can provide a means for selection of transformed cells.

Alternatively a FRET method can be employed, in which, for example, the cell has a targeted cyan fluorescent protein (CFP) having a lipid group or transmembrane protein for targeting to the plasma membrane and has a yellow fluorescent protein (YFP)-reporter, the YFP-reporter can be expressed within the cell or introduced into the cell.

Methods of the invention can be used to detect specific pathway inhibitors capable of inhibiting activation of, or of deactivating, a small GTPase on a specific compartment or membrane. This is useful, for example, if the active small GTPase at one location in the cell is more potent at causing cell transformation and maintaining a tumorigenic state in a particular cell/tissue than that active small GTPase at another location.

When fluorescent reporters are used in methods of the invention, monitoring is performed by fluorescence microscopy using a technique such as wide-field or total internal reflection fluorescence microscopy or fluorescence lifetime imaging or confocal imaging.

Cells for use in methods of the invention may be tumour cells, which may be in vitro model cell lines or primary tumour cells obtained from a patient. Various in vitro model cell lines are suitable for use in methods of the invention, e.g. Cho, Cos, Jurkat-T or HeLa cells.

In preferred embodiments of the invention for detection of membrane bound small GTPase antagonists the cells used are non-serum starved, the advantage of this is that the small GTPase will be active, i.e. GTP-bound due to growth factor receptor-mediated activation of upstream pathways that lead to the GTP-loading of the small GTPase.

To improve the sensitivity of detection, in particular for methods of the invention in which changes in association of the reporter with a membrane bound normal small GTPase are to be monitored, the cells used may overexpress a normal form of the small GTPase and/or may overexpress a GEF specific for that normal small GTPase. Overexpression of a GEF can be used to enhance activation of endogenous normal small GTPase so that transfection and overexpression of a normal small GTPase is not required. Preferably transfection is not required for expression and/or overexpression of the small GTPase. Examples of cells with abnormally increased levels of small GTPase that can be used in methods of the invention include human squamous cell carcinoma cell lines that overexpress normal K-Ras (Hoa M, Davis, S. L., Ames, S. J. and Spanjaard, R. A. Cancer Research (2002) V62: 7154-7156). Hyperactive Ras in cells from patients with neurofibromatosis (DeClue J. E., Papageorge A. G., Fletcher J. A., Diehl S. R., Ratner N., Vass W. C. and Lowy D. R. Cell (1992) V69: 265-273) or tumours with somatic NF1 mutations (Li Y., Bollag G., Clark R., Stevens J., Conroy L., Fults D., Ward K., Friedman E., Samowitz W., Robertson M., Bradley P., McCormick F., White R. and Cawthorne R. Cell (1992) V69: 275-281). For identification of compounds that deactivate oncogenic Ras expression, methods of the invention may be performed using cell types with varying normal:oncogenic ras gene dosage such as squamous and spindle cell carcinomas from mouse skin (Buchmann. A., Ruggeri B., Klein-Szanto A. J. P. and Balmain A. Cancer Research (1991) V51: 4097-4101).

The invention provides an assay for small GTPase activity modulating compounds comprising a method of the invention.

Methods of the invention performed in high throughput format are provided. The invention provides a high throughput assay for a small GTPase activity modulating compounds comprising a method of the invention. High throughput screens using methods of the invention are particularly useful for identifying compounds that inhibit activation or promote deactivation of small GTPases.

The present invention advantageously provides for the use of instrumentation to detect fluorescent or luminescent signals from cells. In preferred methods the detectable marker is fluorescent and monitoring is performed by fluorescence microscopy. Fluorescence microscopy can be performed using wide-field or total internal reflection fluorescence microscopy (TIRF) or fluorescence lifetime imaging or confocal imaging.

In methods of the invention, cells having the fluorescent reporter can be imaged live, or fixed at a given time point, but preferably cells are imaged live, cells are preferably monitored using a HT imaging device, e.g. Amersham INcell analyzer.

High throughput RAPID Method for Detection of Compounds that Inhibit Activation of Ras or Deactivate Active Ras

COS-7 cells are grown in DMEM supplemented with 10% FCS. The day before transfection cells are seeded out on 96-well microplates to obtain a confluency of 50-60% prior to transfection. One well is used as a flat field solution well and does not contain cells. DNA constructs (pcDNA3.1 H-Ras, pEGFP-C3-RBD (Chiu et al (2002) (supra)) and optionally, for a positive control, pCl-neo CAPRI [10] were incubated with Genejuice (Novagen) according to manufacturers instructions and added to the cell medium to transfect (lipofect) cells. In a preferred embodiment COS-7 cells stably expressing H-Ras and GFP-RBD are FACS sorted to select GFP+ cells. Lines displaying clear membrane-localised fluorescence are used in the assay. The cells are incubated for 24 hours at 37° C., 5% CO₂, 95% humidity. The growth medium is carefully removed and 100 μl assay buffer (KH buffer [5 mM HEPES, 10 mM glucose, 25 mM NaHCO₃ 1.2 mM K₂HPO₄, 118 mM NaCl, 4.7 mM KCl, 1.2 mM MgSO₄, 1.3 mM CaCl₂ (pH 7.4)]) warmed to 37° C. is added per well. Cells are transferred to an IN Cell Analyzer 3000 and incubated for 5 minutes at to 37° C., 5% CO₂, 95% humidity. Cells in each well are then read for up to 60 minutes. The test compounds are prepared in assay buffer warmed to 37° C., suitably the final concentration of test compound in the assay is from 2 to 2000 nM, thus the stock solutions of test compound are prepared at 3× strength (e.g. 6 to 6000 nM) and a 50 μl aliquot of stock solution is added per well. For the positive control wells (a triple transfection with pCl-neo CAPRI to express the Ras GAP in H-Ras/GFP-RBD cells), instead of the test compound, a 50 μl aliquot of the agonist ATP (to a final concentration of 50 μM) is added. Cells are transferred to an IN Cell Analyzer 3000 and cells in each well are read for up to 60 minutes. Inhibition of Ras activation, or deactivation of active Ras, is detected by dissociation of the fluorescent GBP-RBD reporter from the plasma and/or Golgi membranes, into the cytosol. This is read as a loss of fluorescent signal from the membrane and/or increase in fluorescent signal in, the cytosol. Data analysis can be performed using the IN Cell Analyzer 3000 Plasma Membrane Spot Analysis module.

In methods of the invention, monitoring can be performed by measuring fluorescence at the region(s) of interest within the cell over time. Association of the reporter with the membrane bound small GTPase and/or decrease of reporter in the cytosol (or vice versa) can be assessed using any suitable algorithm or equation, an example is by calculating the relative translocation parameter (1-Ft/Fo) at one or more time points, wherein Fo is the fluorescence in a region(s) of interest (e.g. cytosol and/or plasma membrane) at the start of monitoring and Ft is fluorescence in a region(s) of interest at a later time point or points.

During monitoring, readings should be made as often as possible, ideally at intervals of less than 10 seconds; the length of time over which monitoring is performed will vary with the nature of the channel being analysed. Monitoring may be performed for time periods of from several seconds, to up to an hour. Suitably, readings may be taken every 5, 10, 15, 20 or 30 seconds over time periods of 5, 10, 20, 30 or 60 minutes. The frequency of readings and time period for monitoring can be experimentally determined and readily optimised for a particular assay, i.e. for particular cells/membrane bound small GTPases.

Monitoring can be performed by measuring cytosolic fluorescence over time as assessed by calculating the relative translocation parameter at one or more time points, 1-Ft_{cyt}/Fo_{cyt}, wherein Fo_{cyt} is the cytosolic fluorescence in the region of interest at the start of monitoring and Ft_{cyt} is the cytosolic fluorescence in the region of interest at a particular time point. A decrease in cytosolic fluorescence results in an increase in relative translocation parameter.

Methods of the invention can be performed in high throughput format.

Methods of the invention are ideal for high throughput screening for compounds capable of modulating a membrane bound small GTPase of interest, for example in 96, 384, or 3456 multiwell plates or other plate formats. In a preferred embodiment, the assay is conducted in a multi well plate format and an instrument is used for monitoring in each well.

The invention provides methods for identification of compounds capable of modulating a membrane bound small GTPase, preferably the methods are performed in high throughput screening (HTS) format, using cells having a reporter derived from an effector of the membrane bound small GTPase (e.g. for a Ras, a fluorescent protein tagged Raf-1-RBD or a derivative thereof specific for the Ras) which is expressed by the cell or introduced into the cell.

In a method of the invention, live whole cells expressing a fluorescent protein-reporter construct, are incubated in the presence of a test compound and the effect of the test compound on the membrane bound small GTPase (inhibition/activation) can be detected; with translocation of the reporter being indicative of modulation of the membrane bound small GTPase.

In a method in which the membrane bound small GTPase of interest is a Ras, and the reporter is Raf-1-RBD, or a derivative thereof specific for active Ras (Ras-GTP), translocation of the reporter from the membrane to the cytosol, detected by monitoring a decrease in fluorescence at the membrane and/or increase in cytosolic fluorescence is indicative that the compound deactivates Ras, or inhibits the activation of Ras.

In preferred methods of the invention, the detectable reporter is genetically encoded, cell lines of interest can be cloned to stably express the detectable reporter, allowing consistency between experiments.

Cells having the fluorescent reporter are imaged live, or fixed at a given time point (but preferably live) preferably using a HT imaging device, e.g., Amersham IN Cell analyzer.

The methods of the invention are ideal for use in HTS to identify new compounds capable of modulating activity of membrane bound small GTPases. The method can be performed using currently available multi-well imaging platforms.

The present invention provides a high throughput screening method for identifying a compound capable of promoting deactivation of a membrane bound active Ras, comprising:
incubating in the presence of a test compound a live cell expressing Ras and a specific reporter thereof, preferably GFP-RBD or a derivative thereof, and
monitoring association of the reporter, preferably GFP-RBD or a derivative thereof, with the membrane bound active Ras

wherein a dissociation of the reporter from the membrane bound active Ras is indicative that the test compound is capable of promoting deactivation of, i.e. switching off, the membrane bound active Ras.

The GFP-RBD reporter is suitable for use in high throughput screening for identification of compounds that inhibit activation of, or deactivate normal active Ras, oncogenic, constitutively active Ras, or normal hyperactive Ras.

In a HTS method for inhibitors of Ras activation or compounds that deactivate Ras, tumour cells expressing hyperactive or oncogenic Ras are transfected with GFP-RBD. Alternatively in vitro model cell lines may be transfected with a desired Ras construct (oncogenic or normal: K-Ras, N-Ras or H-Ras). Inhibition of Ras-GTP is monitored by the dissociation of the fluorescent GFP-RBD reporter from the membrane to the cytosol using a suitable device that can image cellular GFP fluorescence at high resolution on a multi-well format. As Ras-GTP may generate different signalling outcomes from different cellular compartments, e.g. plasma membrane verses Golgi membrane, this methodology allows the determination of selective Ras inhibitors for a specific compartment.

The invention further provides a compound identifiable or identified by a method or assay of any of the preceding claims and the use of such a compound as a medicament. Also provided is the use of such a compound in the manufacture of a medicament for the treatment of the human or animal body, in particular, in the manufacture of a medicament for the treatment of tumours or for the treatment of cancer.

The present invention provides a compound identified or identifiable using a method of the invention capable of modulating the activity of a small GTPase.

The present invention provides a compound, identified or identifiable using a method of the invention, capable of deactivating, i.e. switching off a small GTPase. Preferably the compound is a CAPRI activating peptide, capable of switching off Ras activity, most preferably consisting of or comprising a peptide selected from CVEAWD or RVELWD or a functional analogue, derivative or fragment thereof.

The present invention provides a compound, identified or identifiable using a method of the invention, capable of promoting the activity of a small GTPase. Preferably the compound is a CAPRI inhibiting peptide capable of promoting Ras activity, i.e. activating Ras or maintaining Ras in the active (GTP-bound) state, most preferably consisting of or comprising a peptide selected from SCYPRWNET and KDRNGTSDPFVRV, TRFPHWDEV, RDISGTSDPFARV or a functional analogue, fragment or derivative thereof.

Manipulation of translocation and activation of the Ras GAP CAPRI forms the basis of modulation of Ras GAP activity. CAPRI is inactive in the cytosol but is activated by a mechanism induced by membrane translocation [10]. Without wishing to be bound by any particular theory, it is believed that CAPRI could be locked in an inactive conformation that opens after docking with the membrane, perhaps in association with other proteins. This translocation and activation is dependent on the C2A and C2B domains of CAPRI. Peptides of the invention are capable of interacting with these domains to modulate CAPRI activity and thereby modulate Ras activity.

The Ras GAP RASAL translocates in a Ca²⁺ and C2 domain-dependent manner to the plasma membrane of agonist stimulated cells. It has a conserved GTPase-activating protein-related domain (GRD) and is thought to operate in a similar manner to CAPRI as a Ca²⁺-triggered Ras GAP. Like CAPRI it is believed to interact with a scaffold such as a RACK, and like CAPRI it has a potential pseudo-RACK1 activating peptide sequence in the C2B domain of RASAL. Similarly, the inhibitory peptide sequences C2-2 and C2-4 are highly related to those of CAPRI, indicating that RASAL is capable of a RACK interaction.

CAPRI activating compounds, such as peptides, permit manipulation of GAP activity in treated cells to inhibit normal cellular Ras by the hijacking of endogenous CAPRI protein in a highly specific manner. Thus CAPRI activating compounds such as peptides can be used to activate CAPRI, thereby enhancing the intrinsic GTPase activity of Ras and deactivating active Ras to provide an anti-Ras strategy to for the treatment of tumours that contain hyperactive normal Ras. Manipulating the interaction of CAPRI with RACKs by using compounds, preferably peptides, that mimic the CAPRI-RACK interaction provides a very highly specific means for activation of CAPRI and thus deactivation of Ras, much more so than FTI anti-Ras strategies for example. This strategy is inherently less prone to development of resistance.

The invention provides compounds, such as peptides, or antibodies or fragments thereof, or small molecules capable of activating CAPRI, e.g. by forcing CAPRI to translocate to the plasma membrane and in the process become activated.

Purified CAPRI-activating or CAPRI-inhibiting compounds, e.g. peptides or analogues can be generated in which one or more peptide bonds have been replaced with an alternative type of covalent bond (a ‘peptide mimetic’) resistant to cleavage by peptidases. Such mimetics are well known in the art. Chemical modification whereby charged side-chains of peptides or analogues thereof are blocked can be used to enhance passage of the peptide or analogue through the hydrophobic membrane of the cell.

Mutant Ras GAPs that have very high affinity for Ras-GTP have been proposed as blockers of oncogenic Ras signalling but, until now, there has been no attempt to manipulate endogenous Ras GAPs to deactivate hyperactive Ras. CAPRI-activating peptides provide a mechanism to inhibit normal Ras in tumour cells that have lost Ras GAPs and/or express oncogenes that constitutively activate normal Ras and/or have abnormally high expression of normal Ras. CAPRI activation may block oncogenic Ras signalling through competition with Ras effectors.

“Peptide” and “polypeptide” are used interchangeably herein and refer to a compound made up of a chain of amino acid residues linked by peptide bonds. In analogues such as peptide mimetics, one or more peptide bond(s) may be replaced by an alternative covalent bond. Unless otherwise indicated, the sequence for peptides and analogues thereof is given in the order from the amino terminus to the carboxyl terminus.

A peptide or peptide fragment or analogue thereof is “derived from” a parent peptide or polypeptide if it has an amino acid sequence that is identical or homologous to at least part of the amino acid sequence of the parent peptide or polypeptide. A functional derivative or fragment is a derivative or fragment that modulates CAPRI activity. Particularly preferred are functional peptide or analogue derivatives or fragments thereof that activate CAPRI. CAPRI activation can be determined by several methodologies.

The ability of an activating compound, e.g. a peptide, to induce translocation of CAPRI to the plasma membrane (potentially activating CAPRI) can be monitored by applying cell-permeable compound, e.g. a cell-permeable peptide to live cells expressing GFP-tagged CAPRI imaged by confocal or wide-field microscopy. While this method detects translocation of CAPRI to he membrane, it does not report the activation status of Ras. The GFP-RBD Ras reported can be used in a method of the invention to monitor deactivation of Ras in live cells. Methods of the invention described herein can be used to assess the effect of compounds on Ras in cells transfected with CAPRI. The ability of an activating compound such as a peptide to stimulate CAPRI can be determined by detecting the dissociation of the GFP-RBD from active Ras at the plasma and/or Golgi membrane. This can be performed on both non-transfected cells to analyse the influence of endogenous CAPRI, and on cells overexpressing ectopic CAPRI.

Alternatively, the effect of CAPRI activating peptides can be determined biochemically using a Ras-GTP pull-down assay [10] to measure the deactivation of Ras. Western blotting of cell extracts with phospho-specific antibodies to mitogen-activated protein kinases (MAPKs) prepared after peptide treatment can determine the ability of CAPRI activating peptides to abrogate downstream Ras signalling [10]. However, this method does not permit spatio-temporal analysis of Ras inhibition.

Derivatives may be produced by addition, deletion or substitution of one or more amino acid residues. Preferably one, two or three amino acid residues are substituted. Conservative amino acid substitutions are preferred. Conservative amino acid substitutions are substitutions which do not result in a significant change in the activity or tertiary structure of a selected peptide. Such substitutions typically involve replacing a selected amino acid residue with a different residue having similar physico-chemical properties. For example, substitution of Glu for Asp is considered a conservative substitution since both are similarly-sized negatively-charged amino acids Groupings of amino acids by physico-chemical properties are known to those of skill in the art.

Preferred CAPRI-activating compounds of the invention include peptides consisting of or comprising peptides identified as SEQ ID NOS: 1 and 7, or a functional analogue, derivative or fragment thereof.

Preferred CAPRI-inhibiting compounds of the invention include peptides consisting of or comprising peptides identified as SEQ ID NOS: 3, 5, 9 and 11, or a functional analogue, derivative or fragment thereof.

Peptides of the invention, functional analogues, fragments and derivatives thereof, can be recombinantly produced or chemically synthesised. Peptides of the invention, functional analogues, fragments and derivatives thereof, are preferably small, between 4 and 20 amino acids in length, e.g. 4, 5, 6, 7, 8, 9, 10, 11, 12, 13, 14, 15, 16, 17, 18, 19 or20 amino acids in length, preferably from 4 to 16 amino acids in length. CAPRI activating peptides are preferably from 4 to 10 amino acids in length, most preferably from 4 to 8 amino acids in length. Small peptides are particularly preferred when the peptide is to be transferred into cells; generally, the smaller the peptide, the more readily it can be introduced into a cell.

Also provided are nucleic acid sequences encoding peptides of the invention and functional derivatives and fragments thereof. Suitably these may be provided in an expression vector which can be introduced into a host, e.g. for in vivo expression of the peptide of the invention. Expression constructs may be used for production of peptides, the peptides being isolated for use as therapeutic agents. Alternatively, a construct may be used to deliver the therapeutic peptide, e.g. an expression construct or a viral construct.

Peptides, antibodies, functional analogues, derivatives or fragments thereof of the invention may be chemically modified. Peptides may be linked to transport molecules, e.g. fatty acid molecules such as stearic acid or myristyl acid, this is particularly important for transport of small peptides (4-10 amino acid residues in length) across biological membranes. Thus, chemical modification can be by alkylation using stearation or myrstoylation (Kelemen, B. R., Hsiao, K. and Goueli, S. A. Selective in vivo inhibition of mitogen-activated protein kinase activation using cell-permeable peptides J. Biol. Chem 277, 8741-8748 (2002)). In a preferred aspect peptides and the like are modified by alkylation, as this improves their membrane permeability.

Compounds of the invention and particularly peptides preferably act intracellularly, thus it is important that such compounds can be delivered to the interior of the cell. Peptides can be delivered to cells using known methods such as by transient permeabilisation, or by carrier peptide. A peptide, functional analogue or fragment or derivative thereof can be linked to a moiety effective to facilitate transport across a cell membrane.

Suitable transporters include transport peptides derived from Drosophila antennapedia homeotic transcription factor, the human immunodeficiency virus-TAT protein, the h region of the signal sequence of Kaposi fibroblast growth factor (MTS) and the protein PreS2 of hepatitis B virus (Kelemen, B. R., Hsiao, K. and Goueli, S. A. Selective in vivo inhibition of mitogen-activated protein kinase activation using cell-permeable peptides J. Biol. Chem 277, 8741-8748 (2002)). Lipid-based transfection reagents can be used to deliver peptides and the like, suitable reagents include those described by Zelphati, O. et. al. Intracellular delivery of proteins with a new lipid-mediated delivery system J. Biol. Chem. 276, 35103-35110 (2001).

Peptides can be linked to a second peptide, e.g. a peptide tag, to form a fusion peptide. Suitable peptide tags include hexa-His. The fusion peptides may be capable of binding reactions for example, to attach the peptide, covalently or non-covalently, to a solid support such as a well or bead.

The invention provides a compound according to the invention, preferably a peptide or a functional analogue, derivative or fragment thereof, for use as a medicament.

The invention provides a compound according to the invention, preferably a peptide or a functional analogue, derivative or fragment thereof, for use in the treatment of tumours.

The invention provides the use of a compound according to the invention, preferably a peptide or a functional analogue, derivative or fragment thereof, in the manufacture of a medicament for the treatment of tumours.

The invention provides a method of treatment, in particular of tumours, comprising administration of a compound of the invention to a subject.

The invention provides a composition comprising a compound, according to the invention, preferably a peptide or a functional analogue, derivative or fragment thereof, and a pharmaceutically acceptable carrier or diluent.

The compound or composition according to the invention can be administered by a route selected from intravenous, parenteral, subcutaneous, inhalation, intranasal, sublingual, mucosal, and transdermal.

A compound of the invention, preferably a peptide, or a functional analogue derivative or fragment thereof; more preferably a CAPRI-activating compound, can be administered via parental, subcutaneous, intravenous, intramuscular, intraperitoneal, transdermal, or buccal routes. Alternatively, or concurrently, administration may be by the oral route, or by inhalation. The dosage administered will be dependent upon the age, health, and weight of the recipient, kind of concurrent treatment, if any, frequency of treatment, and the nature of the effect desired.

A composition comprising a compound of the invention, in particular a peptide, or functional analogue derivative or derivative thereof, more preferably a CAPRI-activating compound, may contain suitable pharmaceutically acceptable carriers such as excipients and auxiliaries which facilitate processing of the active compounds into preparations which can be used pharmaceutically for delivery to the site of action. Suitable formulations for parenteral administration include aqueous solutions of the active compounds, for example in saline. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides. Aqueous injection suspensions may contain substances which increase the viscosity of suspension include, for example, sodium carboxymethyl cellulose, sorbitol, and/or dextran.

Optionally, the suspension may also contain stabilizers. Liposomes can also be used to encapsulate the agent for delivery.

A formulation for systemic administration according to the invention may be formulated for enteral, parenteral or topical administration. Suitable formulations for oral administration include hard or soft gelatin capsules, pills, tablets, including coated tablets, elixirs, suspensions, syrups or inhalations and controlled release forms thereof. Suitable formulations for administration by inhalation include metered dose inhalers and dry powder devices. For nasal absorption aqueous and non-aqueous suspensions or dry powders may be used. For local treatment of a tumour mass a biopolymer system for delivery of a CAPRI-activating compound may be implanted in close proximity (Folkman et al., U.S. Pat. No. 4,164,560).

The invention provides a method for identification of a compound, effective to modulate CAPRI activity, comprising contacting CAPRI with a test compound and determining if CAPRI activity is modulated.

The invention provides a method for identification of a compound, effective to activate CAPRI, comprising contacting an inactive form of CAPRI with a test compound and determining if CAPRI is activated. A compound identified or identifiable by such methods is provided also.

The invention provides an in vivo CAPRI assay.

The screening of small molecule libraries using a GFP-CAPRI translocation assay (see FIG. 3) permits discovery of novel compounds that mimic the activating peptides and trigger CAPRI-RACK interaction to activate the GAP activity of CAPRI.

Equipment such as an IN Cell Analyser (Amersham Biosciences) can be used to perform a HTS on a multi-well format of activating peptides designed around the putative CAPRI pseudo-RACK binding site using multiple peptides designed with alternative carrier peptide sequences. Translocation of GFP-CAPRI from the cytosol to the plasma membrane can be assessed. Screens can be performed for small molecule activators of CAPRI using compound libraries.

The invention also provides method of prophylaxis or curative treatment, in particular of tumours, comprising administration of a compound of the invention, CAPRI-activating compound, most preferably a CAPRI-activating peptide to a patient.

### LIST OF FIGURES

FIG. 1. Molecular architecture of the GAP1 family. Percentages indicate identity with CAPRI.

FIG. 2. Alignment of β1-6 of the PKCβ C2B domain with CAPRI and GAP1^{m} (PKC is type I topology). Boxed regions indicate highly conserved RACK binding sequences identified in PKCβ.

FIG. 3. Expression of GFP-C2B (LEFT) compared with GFP-CAPRI (right, 0 s, 30 s and 60 s) in HeLa cells 24 hrs after transient transfection. Live imaging by confocal microscopy.

FIG. 4. Expression of GFP-C2A/C2B in COS-7 cells imaged by live confocal microscopy.

FIG. 5. Transcript distribution (semi-quantitative) Top panel adult human tissues: 1—brain, 2—heart, 3—kidney, 4—lung, 5—pancreas, 6—placenta, 7—skeletal muscle, 8—300 pg CAPRI cDNA. Bottom panel foetal human tissues: 1—brain, 2—heart, 3—kidney, 4—liver, 5—lung, 6—skeletal muscle, 7—spleen, 8—thymus, 10—1 pg CAPRI cDNA, 11—300 pg CAPRI cDNA.

FIG. 6. CAPRI rapidly and specifically deactivates Ras at the plasma membrane in CHO cells stably expressing CAPRI after ATP stimulation. GFP-RBD is localised to the plasma membrane and endomembranes in non-starved, H-Ras transfected CHO.T cells. Addition of ATP (50 μM) to stimulate the release of store Ca²⁺ leads to rapid deactivation of Ras at the plasma membrane which is manifested by the exclusive dissociation of GFP-RBD from the plasma membrane. No such dissociation is seen in parental CHO.T cells.

FIG. 7. Use of the RAPID assay (Ras Activity Probe for Inhibitor Detection) to demonstrate the agonist-dependent activation of CAPRI. CAPRI rapidly and specifically deactivates Ras at the plasma membrane in COS cells ectopically expressing CAPRI, H-Ras and GFP-RBD after 50 μM ATP stimulation. There is no detectable deactivation in cells transfected with H-Ras and GFP-RBD only, or by expression of GAP-dead CAPRI (R47S mutant) under the same conditions and cell stimulation. Dissociation of the RBD from the plasma membrane is expressed as the inverse relative change in fluorescence intensity of a cytosolic ROI and this trace is shown.

FIG. 8. CAPRI rapidly and specifically deactivates Ras at the plasma membrane in COS cells ectopically expressing CAPRI, H-Ras and GFP-RBD (black trace) after 50 μM ATP stimulation. There is no detectable deactivation in cells transfected with H-Ras and GFP-RBD only (grey trace), or by expression of GAP-dead CAPRI (R47S mutant) under the same conditions and cell stimulation. Dissociation of the RBD from the plasma membrane is expressed as Relative Dissociation=maximal pixel intensity of cytosolic ROI (usually at time zero) divided by the cytosolic ROI at time x during the experiment. Ratio of transfected DNA is 1:1:1 for H-Ras, GFP-RBD and CAPRI. Average n=6 experiments from n=9 single cells for H-Ras, GFP-RBD and CAPRI transfected cells (black trace)±standard deviation from the mean. Average n=3 experiments, n=10 single cells for H-Ras and GFP-RBD transfected control cells (grey trace).

FIG. 9. CAPRI rapidly and specifically deactivates Ras at the plasma membrane in HeLa cells ectopically expressing CAPRI, H-Ras and GFP-RBD after 100 μM histamine stimulation. There is no detectable deactivation in cells transfected with H-Ras and GFP-RBD only, or by expression of GAP-dead CAPRI (R47S mutant) under the same conditions and cell stimulation. Dissociation of the RBD from the plasma membrane is expressed as Relative Dissociation=maximal pixel intensity of cytosolic ROI (usually at time zero) divided by the cytosolic ROI at time x during the experiment. Ratio of transfected DNA is 1:1:1 for H-Ras, GFP-RBD and CAPRI. Average n=2 experiments from n=4 single cells for H-Ras, GFP-RBD and CAPRI transfected cells±standard deviation from the mean.

### EXAMPLES

Homology

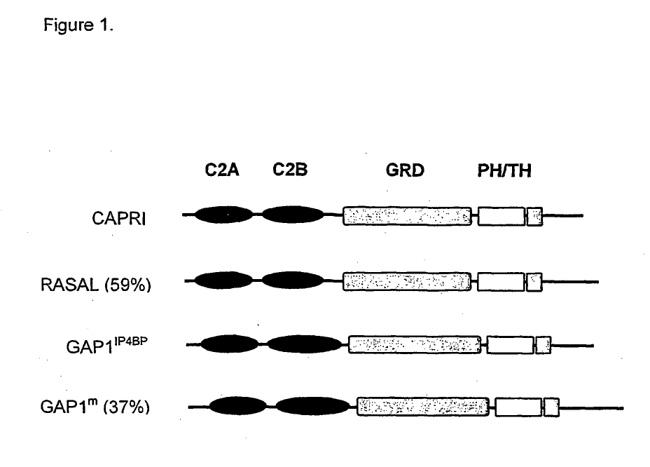

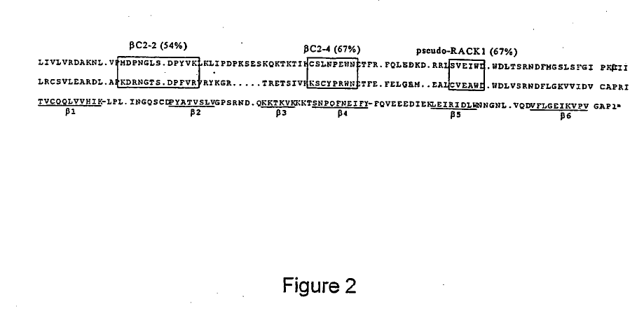

The molecular architecture of the GAP1 family is provided in FIG. 1, the percentages indicate identity with human CAPRI. Outside the GAP1 family, the CAPRI C2B domain has highest identity with PKCβ of all known C2 domains; this is particularly high within PKC regions demonstrated to interact with RACK1 (receptor for activated C-kinase) [10, 11]. FIG. 2 provides an alignment of β1-6 of the PKCβ C2B domain with CAPRI and GAP1^{m} (PKC is type I topology). Highly conserved RACK binding sequences identified in PKCβ are indicated as boxed sections. The βC2-4 and βC2-2 PKC regions have 67% and 54% identity with CAPRI, respectively. The pseudo-RACK binding site in loop 3 of the C2B domain of CAPRI is also highly conserved between CAPRI and PKCβ. The C2A and C2B domains in RASAL are highly homologous to CAPRI and, like CAPRI, RASAL contains five critical aspartate residues per C2 domain that are known to be required for high-affinity Ca²⁺/phospholipids-binding in other Ca²⁺ sensors such as PKCβ.

CAPRI/RACK Interaction

The CAPRI/RACK interaction can be analysed in vitro and in vivo. To determine if GFP-C2B is interacting with RACK1, the C2 domain and endogenous RACK1 can be immunoprecipitated with commercial GFP and RACK1 antibodies. In addition, GST-C2B can be immobilized to a glutathione sopharose column and recombinant RACK1 applied. If a RACK1-like protein, rather than RACK1 itself, associates with the CAPRI C2B domain then this can be tested indirectly, because regions within the PKCβ C2 domain that bind to RACK1 have been mapped and used to develop inhibitory and activating PKC peptides. Equivalent CAPRI inhibitory and activating peptides can be tested for inhibition of translocation and activation of CAPRI respectively.

Peptides are tested for their ability to modulate CAPRI activity by live cell imaging to determine if activating peptides can induce the translocation of GFP-CAPRI to the plasma membrane. If endogenous CAPRI is activated by peptides then the level of Ras-GTP in the cell can be tested by a Ras pull-down assay [10]. In non-starved cells peptide activation of CAPRI will lead to a decrease in Ras-GTP levels. CAPRI activation can also be assayed downstream of Ras by analysing MAPK activation using Western blotting of post-stimulation cell lysates with phosphor-specific antibodies to activated p44 and p42 MAPKs [10]. CAPRI activating compounds e.g. peptides would be predicted to deactivate MAPK signalling in non-starved cells.

The peptides chosen for investigation included the CAPRI activating peptide CVEAWD (pseudo-RACK1) (SEQ ID NO:1) and CAPRI inhibitory peptides KDRNGTSDPFVRV (C2-2) (SEQ ID NO: 3) and SCYPRWNET (C2-4) SEQ ID NO: 4).

Localisation of C2 Domains

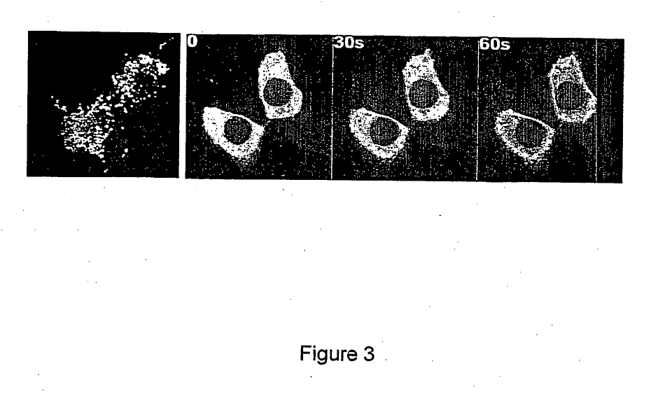

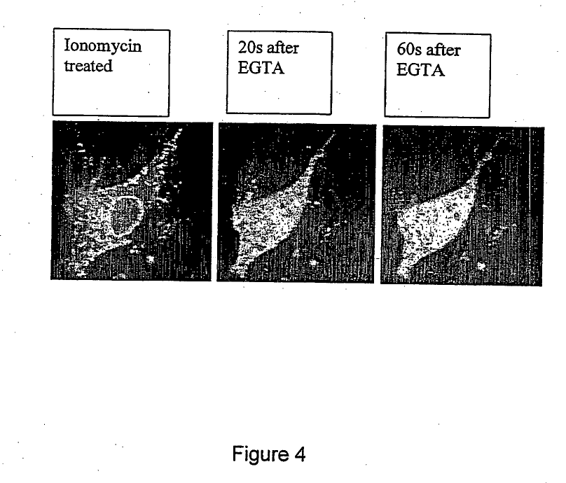

In PKC, the RACK binding site is only exposed after interactions with Ca²⁺/phospholipid, so that the site of RACK interaction is already exposed in the isolated C2 domain. This is interesting with respect to CAPRI because the GFP-C2B domain forms punctate structures concentrated beneath the plasma membrane in resting HeLa cells, whereas the C2A, C2A/C2B, ΔC2A-CAPRI and full-length CAPRI are cytosolic (FIG. 3). Furthermore, the GFP-C2A/C2B chimera forms punctate vesicular structures under the plasma membrane after ionomycin stimulation, and this association is reversible after Ca²⁺ buffering (FIG. 4).

GFP-CAPRI Translocation Assay

The ability of the activating peptide to induce translocation of CAPRI to the plasma membrane can be monitored by applying cell-permeable peptide to live cells expressing GFP-tagged CAPRI imaged by confocal or wide-field microscopy. Experiments were performed to assess expression of GFP-C2B compared with GFP-CAPRI in HeLa cells 24 hrs after transient transfection by lipofection. Live imaging was performed by confocal microscopy. The results are shown in FIG. 3, the image for GFP-C2B is shown on the left hand side, GFP-CAPRI images at 0 s, 30 s and 60 s are shown on the right. Cells expressing GFP-CAPRI were stimulated with carbachol to mobilise intracellular Ca²⁺, time=seconds after stimulation. No detectable effect on GFP-C2B localisation was found (data not shown). CAPRI activating peptides induce the translocation of GFP-CAPRI to the plasma membrane in the absence of agonist stimulation.

Experiments were performed to assess expression of GFP-C2A/C2B domain COS-7 cells imaged by live confocal microscopy. Cells were transfected with GFP-C2A/C2B vector by lipofection and imaged 24 hours later. As shown in FIG. 4, cells were stimulated by the Ca²⁺ onophore ionomycin to induce redistribution of cytosolic protein. Nuclear GFP-C2A/C2B is diffusely localised throughout the nucleoplasm before stimulation (due to size of the fusion protein). This translocates to the inner nuclear membrane without forming a punctate distribution in the nucleoplasm, suggesting that the cytosolic interactions are specific. Application of 5 μM EGTA to the extracellular medium, which buffers intracellular Ca²⁺, causes rapid dissociation of the GFP-C2A/C2B chimera from the membrane into the cytosol and nucleoplasm. The stimulated association/disassociation of these structures are probably due to a specific interaction with endogenous entities near the plasma membrane and within the perinuclear region, rather than non-specific aggregation (see 20 second image).

The C2A/C2B protein may be physiologically relevant since two cDNAs from murine 13 day embryo head (accession number AK014220 GenBank at NCBI) and adult retina (accession number AK044762 GenBank at NCBI) encode a splice variant of CAPRI of just the C2A and C2B domains. This is due to alternative splicing resulting in premature termination because of a retained intron between exon 10 and 11. in the short variant (data unpublished). In theory the splice variant would act as a dominant negative. Expression of this CAPRI variant in human and mouse tissues is being investigated.

HTS using GFP-RBD to Identify Compounds that Deactivate Ras

A GFP-tagged peptide sequence from the Ras-binding domain of Raf (amino acids 51-131 of Raf-1) has high affinity for Ras-GTP but not Ras-GDP. As a consequence in non-starved cells this reporter for active Ras is concentrated at the plasma membrane and on the Golgi membrane, sites where Ras is activated in live cells. This reporter is used in a novel assay for the activation of CAPRI. Translocation of CAPRI by a Ca²⁺ signal from the cytosol to the plasma membrane activates the GAP activity of CAPRI leading to the turnover of Ras-GTP. This causes the dissociation of the GFP-RBD from the plasma membrane, indicating the enhancement of Ras GTPase activity by the action of CAPRI. The kinetics and degree of Ras deactivation can be measured by monitoring the pixel intensity within a defined region of the cytosol (a region of interest at least 10% of the 2D cytosolic image). These experiments have been performed successfully in COS-7 fibroblast co-transfected with H-Ras, CAPRI and GFP-RBD and stimulated with ATP to generate Ca²⁺ signals. The GFP-RBD has also been used to determine the effect of endogenous CAPRI knockdown by RNA interference. In Jurkat T cells after engagement of the T cell receptor, or in HeLa cells stimulated with epidermal growth factor, CAPRI knockdown leads to the sustained activation of Ras at the plasma membrane compared to control cells. This clearly demonstrates that endogenous CAPRI has a primary role in controlling the activation status of Ras by agonists that stimulate Ca²⁺ signalling Bivona et al (August 2003), Nature, 424, 694-8.

Transcript Distribution (Semi-Quantitative)

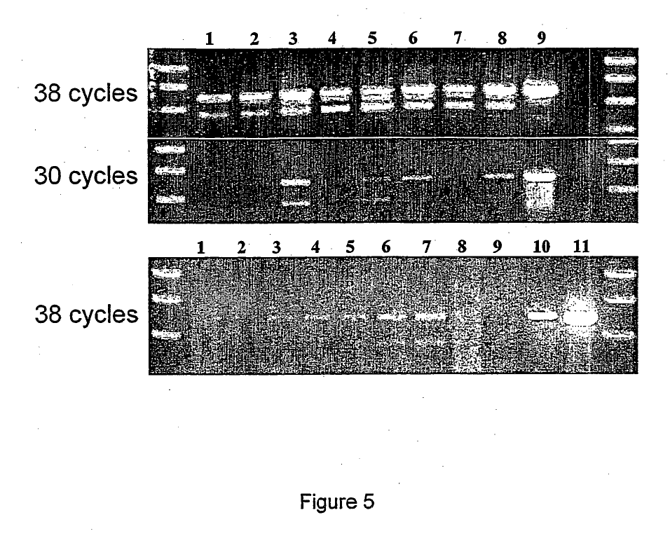

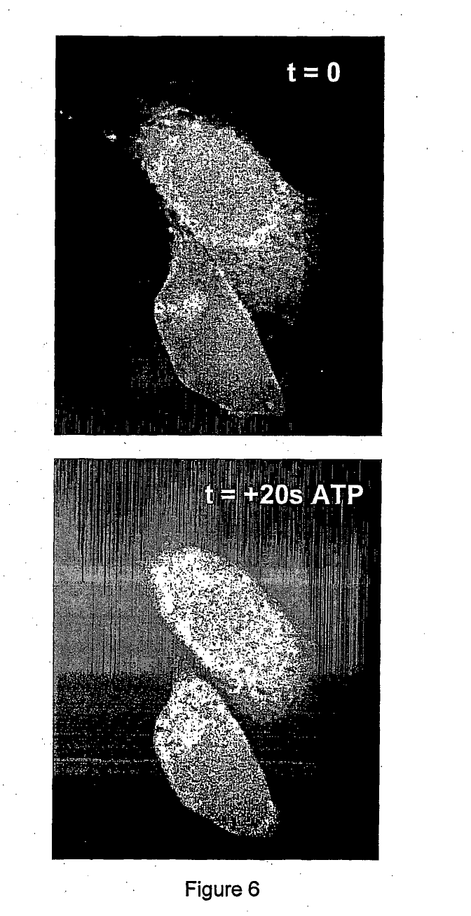

PCR of Clontech MTN cDNA panels was performed to detect full-length CAPRI (upper band) or CAPRI S (PH domain splice variant; lower band, data unpublished). In FIG. 5, the top panel (38 and 30 PCR cycles) is adult human tissues: 1—brain, 2—heart, 3—kidney, 4—lung, 5—pancreas, 6—placenta, 7—skeletal muscle, 8—300 pg CAPRI cDNA. In the bottom panel (38 PCR Cycles) the tissues were of foetal origin: 1—brain, 2—heart, 3—kidney, 4—liver, 5—lung, 6—skeletal muscle, 7—spleen, 8—thymus, 10—1 pg CAPRI cDNA, 11—300 pg CAPRI cDNA.

The results demonstrate that CAPRI is widely expressed. Thus CAPRI activating compounds such as peptides can be used as an anti-Ras strategy, e.g. to treat tumours that contain hyperactive normal Ras in a wide variety of tissues.

RAPID (Ras Activity Probe for Inhibitor Detection) Method for Detection of Compounds that Inhibit Activation of Ras or Deactivate Active Ras

COS-7 cells were grown in DMEM supplemented with 10% FCS. The day before transfection cells were seeded out on 6-well tissue culture plates containing 22 mm circular glass coverslips to obtain a confluency of 50-60% prior to transfection. DNA constructs (pcDNA3.1 H-Ras, pEGFP-C3 RBD and optionally pCl-neo CAPRI) were incubated with Genejuice (Novagen) according to manufacturers instructions and added to the cell medium to transfect (lipofect) cells. 24 hours later coverslips were mounted in a heated stage (37° C.) in KH buffer [5 mM HEPES, 10 mM glucose, 25 mM NaHCO₃ 1.2 mM K₂HPO₄, 118 mM NaCl, 4.7 mM KCl, 1.2 mM MgSO₄, 1.3 mM CaCl₂ (pH 7.4)]. Nipkow confocal microscopy was performed using a PerkinElmer UltraView LCI system to image GFP. For rapid mixing of agonist in positive control reactions (a triple transfection with pCl-neo CAPRI to express the Ras GAP in H-Ras/GFP-RBD cells) ATP (50 μM) was added at a desired time point in a large (5 ml) volume with excess media removed by vacuum line. Test compounds were added at appropriate dilutions and the effect on the association of GFP-RBD reporter with membrane bound active Ras was monitored over time for a period of up to 60 minutes after addition of the test compound. Inhibition of Ras activation, or deactivation of active Ras, was detected by dissociation of the fluorescent GBP-RBD reporter from the plasma and/or Golgi membranes, into the cytosol. This was seen as a loss of fluorescent signal from the membrane/increase in fluorescent signal in the cytosol.

Use of the RAPID Assay (Ras Activity Probe for Inhibitor Detection) to Demonstrate the Agonist-Dependent Activation of CAPRI

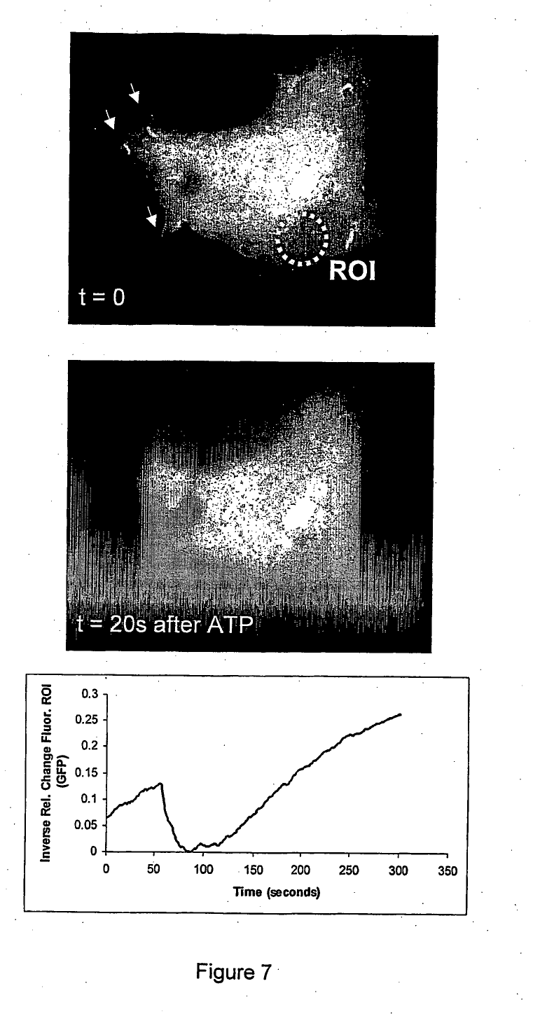

Non-starved COS-7 cells transiently transfected with H-Ras, GFP-RBD and CAPRI were imaged every 1.4 seconds before (t=0) and after (t=20 secs) stimulation with 50 μM ATP to mobilise intracellular Ca²⁺. As shown in FIG. 7, prior to stimulation the GFP-RBD reports significant Ras-GTP in the Golgi and in ruffles at the plasma membrane (arrow heads) demonstrating that CAPRI is inactive (FIG. 7). Stimulation leads to the rapid dissociation of the GFP-RBD exclusively from the plasma membrane and loss of membrane fluorescence with a concurrent increase in cytosolic fluorescence intensity. This indicates that CAPRI specifically deactivates Ras at the plasma membrane and not the Golgi, and is consistent with previous work showing the exclusive translocation of CAPRI to the plasma membrane (Lockyer, P. J, Kupzig, S. and Cullen, P. J. Curr Biol (2001) V11:981-986).

The change in location of the reporter GFP-RBD probe can be measured by highlighting a region of interest (ROI) in the plasma membrane, in the cytosol, or expressed as a ratio of the two. In this example the increase in cytosolic fluorescence intensity has been measured by highlighting a ROI of least 10% of the cytosolic area. The inverse relative change in fluorescence intensity at a given time point is expressed as, 1−(cytosolic fluorescence intensity/maximum experimental cytosolic fluorescence intensity)

In the example above there is a steady rate of bleaching due to the fast imaging applied but speed of acquisition may be of limited importance for a HTS on a multi-well format.

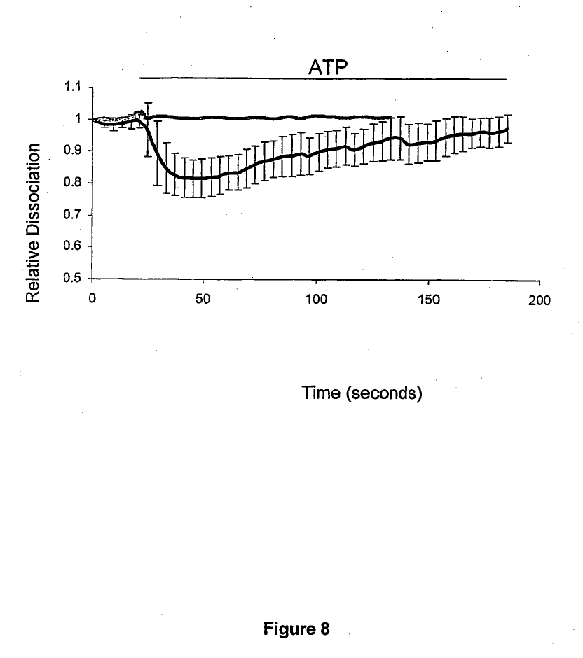

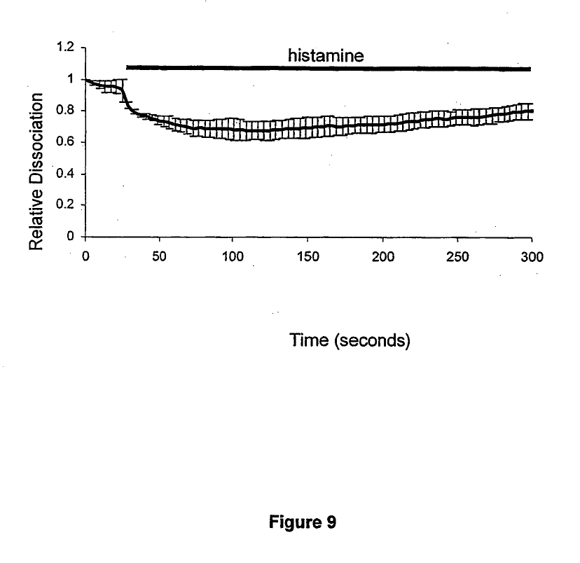

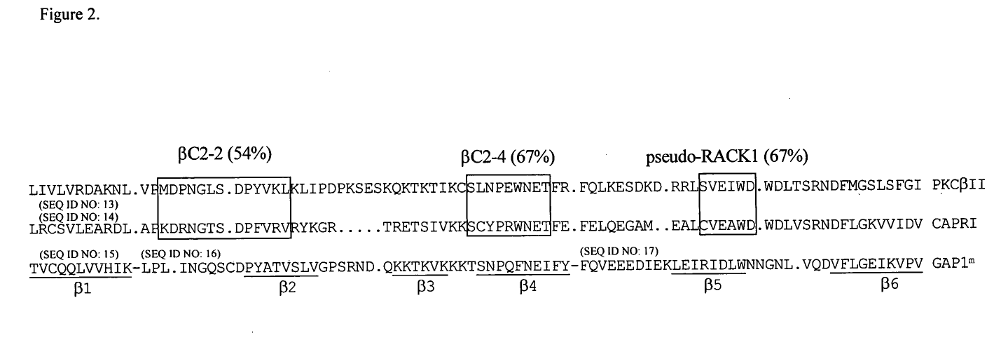

Non-starved COS-7 or HeLa cells transiently transfected with H-Ras, GFP-RBD and CAPRI were imaged every 4 seconds before and after stimulation with 50 μM ATP (COS-7) or 100 μM histamine (HeLa) to mobilise intracellular Ca²⁺. As shown in FIGS. 8 and 9, stimulation leads to the rapid dissociation of the GFP-RBD from the plasma membrane, loss of membrane fluorescence with a concurrent increase in cytosolic fluorescence intensity expressed as Relative Dissociation parameter. Relative Dissociation=maximal pixel intensity of cytosolic ROI (usually at time zero) divided by the cytosolic ROI at time x during the experiment. Ratio of transfected DNA is 1:1:1 for H-Ras, GFP-RBD and CAPRI. There is zero photobleaching due to a relatively slow rate of acquisition.

### REFERENCES

1. Barbacid, M., Ras Genes. Annual Review of Biochemistry, 1987. 56: p. 779-827.

2. Campbell, S. L., et al., Increasing complexity of Ras signalling. Oncogene, 1998. 17(11): p. 1395-1413.

3. Bos, J. L., Ras Oncogenes in Human Cancer—a Review. Cancer Research, 1989. 49(17): p. 4682-4689.

4. Cichowski, K. and T. Jacks, NF1 tumour suppressor gene function: Narrowing the GAP. Cell, 2001. 104(4): p. 593-604.

5. Nori, M., et al., Inhibition of V-Src-Induced Transformation by a Gtpase-Activating Protein. Molecular and Cellular Biology, 1991. 11(5): p. 2812-2818.

6. Gibbs, J. B., et al., Modulation of Guanine-Nucleotides Bound to Ras in Nih3t3 Cells by Oncogenes, Growth-Factors, and the Gtpase Activating Protein (Gap). Journal of Biological Chemistry, 1990. 265(33): p. 20437-20442.

7. Smith, M. R., S. J. Degudicibus, and D. W. Stacey, Requirement for C-Ras Proteins During Viral Onco gene Transformation. Nature, 1986. 320(6062): p. 540-543.

8. Hoa, M., et al., Amplification of Wild-Type K-ras Promotes Growth of Head and Neck Squamous Cell Carcinoma. Cancer Res, 2002. 62(24): p. 7154-7156.

9. Cullen, P. J. and P. J. Lockyer, Integration of calcium and Ras signalling. Nature Reviews Molecular Cell Biology, 2002. 3(5): p. 339-348.

10. Lockyer, P. J., S. Kupzig, and P. J. Cullen, CAPRI regulates Ca2+-dependent inactivation of the Ras-MAPK pathway. Current Biology, 2001. 11(12): p. 981-986.

11. Ron, D., et al., Cloning of an Intracellular Receptor for Protein-Kinase-C—a Homolog of the Beta-Subunit of G-Proteins. Proceedings of the National Academy of Sciences of the United States of America, 1994. 91(3): p. 839-843.

12. Ron, D., J. H. Luo, and D. Mochlyrosen, C2 Region-Derived Peptides Inhibit Translocation and Function of Beta-Protein Kinase-C in-Vivo. Journal of Biological Chemistry, 1995. 270(41): p. 24180-24187.

13. Ron, D. and D. Mochlyrosen, An Autoregulatory Region in Protein-Kinase-C—the Pseudoanchoring Site. Proceedings of the National Academy of Sciences of the United States of America, 1995. 92(2): p. 492-496.

14. Rodriguez, M. M., et al., RACK1, a protein kinase C anchoring protein, coordinates the binding of activated protein kinase C and select pleckstrin homology domains in vitro. Biochemistry, 1999. 38(42): p. 13787-13794.

15. Dorn, G. W. and D. Mochly-Rosen, Intracellular transport mechanisms of signal transducers. Annual Review of Physiology, 2002. 64: p. 407-429.

16. Dorn, G. W., et al., Sustained in vivo cardiac protection by a rationally designed peptide that causes epsilon protein kinase C translocation. Proceedings of the National Academy of Sciences of the United States of America, 1999. 96(22): p. 12798-12803.

### Sequence Listing Information

SEQ ID NO: 1 CVEAWD

CAPRI activating peptide (pseudo-RACK1)

SEQ ID NO: 2 coding sequence CVEAWD

TGCGTGGAGGCCTGGGAC (667-684 of Genbank AY029206)

SEQ ID NO: 3 KDRNGTSDPFVRV

CAPRI inhibitory peptide (C2-2)

SEQ ID NO: 4 coding sequence KDRNGTSDPFVRV

AAGGACCGCMTGGCACATCTGACCCCTTCGTCCGAGTG (520-558 of Genbank AY029206)

SEQ ID NO: 5 SCYPRWNET

CAPRI inhibitory peptide (C2-4)

SEQ ID NO: 6 coding sequence SCYPRWNET

TCATGCTACCCACGCTGGMTGAGACG (601-627 of Genbank AY029206)

SEQ ID NO: 7 RVELWD

RASAL activating peptide (pseudo-RACK1)

SEQ ID NO: 8 coding sequence RVELWD

CGGGUGGAGCUCUGGGAC (882-899 of Genbank NM_{—}004658)

SEQ ID NO: 9 TRFPHWDEV

RASAL inhibitory peptide (C2-4)

SEQ ID NO: 10 coding sequence TRFPHWDEV

ACUCGCUUCCCGCACUGGGAUGMGUG (816-842 of GenBank NM_{—}004658)

SEQ ID NO: 11 RDISGTSDPFARV

RASAL inhibitory peptide (C2-4)

SEQ ID No: 12 coding sequence RDISGTSDPFARV

GCUCCCAGAGACAUCUCUGGCACAUCUGACCCAUUUGCACGUGUG (729-773 of GenBank NM_{—}004658)

What is claimed is:

1-59. (canceled)

60. A method for identifying a compound capable of promoting deactivation or inhibiting activation of a membrane bound active small GTPase, comprising:
incubating in the presence of a test compound a live cell expressing the membrane bound small GTPase and comprising an active small GTPase specific reporter comprising a small GTPase specific binding moiety and a detectable marker moiety; and,
monitoring association of the reporter with the membrane bound small GTPase, wherein a dissociation of the reporter from the membrane bound small GTPase is indicative that the test compound is capable of promoting deactivation of the membrane bound active small GTPase.

61. The method according to claim 60 wherein the small GTPase is bound to at least one of the plasma membrane, Golgi apparatus membrane, endomembrane, mitochondrial membrane, outer nuclear membrane, inner nuclear membrane, endoplasmic reticulum, sarcoplasmic reticulum, a membrane of transport, and secretory vesicles.

62. The method according to claim 60 wherein the membrane bound small GTPase is a Ras superfamily GTPase.

63. The method according to claim 60 wherein the membrane bound small GTPase is a Ras, Rho, Ran, Arf/Sar1, or Rab/YPT1 subfamily GTPase.

64. The method according to claim 60 wherein the small GTPase is a hyperactive or a constitutively active mutant small GTPase.

65. The method according to claim 60 wherein the small GTPase is a Ras.

66. The method according to claim 60 wherein the membrane bound small GTPase is one or more Ras GTPase, selected from the group consisting of H-Ras, K-Ras and N-Ras.

67. The method according to claim 60 wherein the small GTPase is a hyperactive or oncogenic Ras.

68. The method according to claim 60 wherein the small GTPase specific binding moiety is a peptide derived from an effector of the small GTPase.

69. The method according to claim 60 wherein the small GTPase specific binding moiety is a peptide derived from an effector of the small GTPase having one or more point mutations that increase the affinity of the peptide for the small GTPase relative to the affinity of the wild type effector for the small GTPase.

70. The method according to claim 60 wherein the small GTPase is a Ras and the small GTPase-specific binding moiety is an active-Ras-specific-binding moiety.

71. The method according to claim 70 wherein the active-Ras specific binding moiety is Raf-1-RBD, or a derivative thereof.

72. The method according to claim 60 wherein the reporter is a reporter protein.

73. The method according to claim 60 wherein the detectable marker moiety is selected from a luminescent protein and a fluorescent protein.

74. The method according to claim 73 wherein monitoring is performed by fluorescence microscopy.

75. The method according to claim 73 wherein monitoring is performed by fluorescence microscopy using a method selected from the group consisting of wide-field fluorescence microscopy, total internal reflection fluorescence microscopy, fluorescence lifetime imaging and confocal imaging.

76. The method according to claim 60 wherein the cell is selected from the group consisting of a tumour cell, a primary tumour cell and a cell from an in vitro model cell line.

77. A method for identifying a compound capable of promoting deactivation of a membrane bound active Ras, comprising:
incubating in the presence of a test compound a live cell expressing Ras and a specific reporter comprising GFP-RBD or a derivative thereof; and,
monitoring association of the reporter with the membrane bound active Ras, wherein a dissociation of the reporter from the membrane bound active Ras is indicative that the test compound is capable of promoting deactivation of the membrane bound active Ras.

78. The method according to claim 60 performed in high throughput format.

79. The method according to claim 77 performed in high throughput format.

80. A high throughput screening method for identifying a compound capable of promoting deactivation of a membrane bound active Ras, comprising:
incubating in the presence of a test compound a live cell expressing Ras and a specific reporter comprising GFP-RBD or a derivative thereof; and,
monitoring association of the reporter with the membrane bound active Ras, wherein a dissociation of the reporter from the membrane bound active Ras is indicative that the test compound is capable of promoting deactivation of the membrane bound active Ras.
